(19) United States
(12) Patent Application Publication
(10) Pub. No.: US 2025/0313526 A1
(43) Pub. Date: Oct. 9, 2025

(54) PYRETHROID COMPOUND CONTAINING DOUBLE BONDS AND CYANO GROUPS, SYNTHESIS METHOD THEREFOR, AND APPLICATION THEREOF

(71) Applicant: Jiangsu Yangnong Chemical Co., Ltd., Yangzhou, Jiangsu (CN)

(72) Inventors: Xiaoju Wu, Yangzhou, Jiangsu (CN); Youfa Jiang, Yangzhou, Jiangsu (CN); Baolin Wang, Yangzhou, Jiangsu (CN); Chengmei Huang, Yangzhou, Jiangsu (CN); Wei Jia, Yangzhou, Jiangsu (CN); Yang LV, Yangzhou, Jiangsu (CN)

(21) Appl. No.: 18/877,417

(22) PCT Filed: Jan. 19, 2023
(86) PCT No.: PCT/CN2023/073129

§ 371 Date: Dec. 20, 2024

(30) Foreign Application Priority Data
Feb. 22, 2022 (CN) 202210160506.3

(51) Int. Cl. C07C 255/31; A01N 53/00; A01P 7/04; A01P 17/00; C07C 253/30; C07C 253/34

(52) U.S. Cl. CPC C07C 255/31; A01N 53/00; A01P 7/04; A01P 17/00; C07C 253/30; C07C 253/34; C07C 2601/02

(57) ABSTRACT

Disclosed are a pyrethroid compound containing a cyano group and a double bond, a synthesis method therefor and an application thereof. In the synthesis method, t-butyl 3-formyl-2,2-dimethyl-cyclopropanoate and cyanomethyl phosphonate are used as raw materials, undergo Witting-Horner reaction, undergo column chromatography to obtain t-butyl (Z)-3-(2-cyanovinyl)-2,2-dimethyl-cyclopropanoate ester, undergo deprotection to obtain (Z)-3-(2-cyanovinyl)-2,2-dimethyl-cyclopropanoic acid, undergo acyl chlorination reaction to produce (Z)-3-(2-cyanovinyl)-2,2-dimethyl-cyclopropanoyl chloride, and are esterified with corresponding alcohol to obtain the pyrethroid compound. The compound can be used as an insecticide against pests such as mosquitoes, flies, German cockroaches and the like.

### TECHNICAL FIELD

The present invention relates to a pyrethroid compound useful for killing insects, and in particular to a method for synthesizing a pyrethroid compound containing a cyano group and a double bond, and an application thereof in prevention and control of mosquitoes, flies and other pests. The present invention belongs to the field of chemical synthesis technology.

### BACKGROUND ART

Pyrethroids are active ingredients in mosquito-repellent incense and aerosols, and are mainly used to prevent and control household pests such as mosquitoes and flies. As people have long been using pyrethroid products, mosquitoes have varying degrees of resistance to most products available on the market. Thus, developing a pyrethroid with a novel structure would be helpful to alleviate the problem of resistance, and prolong the service life of such type of insecticides.

Momfluorothrin (US2012/29227, EP2241551) is a new pyrethroid insecticide developed by Sumitomo Corporation of Japan. It has good contact-killing effect on insects and spiders, but there are few reports on its use for household pests. After testing, it was found that while Momfluorothrin as an aerosol in a cylindrical container had good knockdown activity against mosquitoes, it exhibited relatively poor activity as mosquito-repellent incense. This may be attributed to the fact that its nitrogen-containing structure leads to a lower vapor pressure, making it difficult to volatize through heating to achieve contact-killing effect.

By simplifying the structure of Momfluorothrin, the present invention increases the vapor pressure of the compound, and further improves its effect as mosquito-repellent incense, thereby laying the foundation for the application of a pyrethroid compound containing a cyano group and a double bond for mosquito prevention and control.

### SUMMARY OF THE INVENTION

The technical problem to be solved by the present invention is to address the deficiencies in the prior art by providing a pyrethroid compound containing a cyano group and a double bond, a synthesis method therefor and an application thereof. The compound is used as an insecticide for prevention and control of household pests such as mosquitoes, flies and German cockroaches.

To achieve the above objective, the present invention employs the following technical solution: The present invention first provides a pyrethroid compound containing a cyano group and a double bond, represented by formula I:

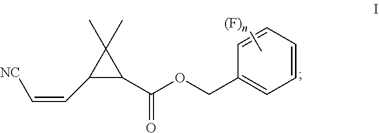

wherein n is 3 or 4.

In the above technical solution, when n is 3, the compound is 2,3,5-trifluorobenzyl (Z)-3-(2-cyanovinyl)-2,2-dimethylcyclopropanecarboxylate or 2,3,6-trifluorobenzyl (Z)-3-(2-cyanovinyl)-2,2-dimethylcyclopropanecarboxylate.

In the above technical solution, when n is 4, the compound is 2,3,5,6-tetrafluorobenzyl (Z)-3(2-cyanovinyl)-2,2-dimethylcyclopropanecarboxylate.

In the above technical solution, the compound is preferably 2,3,5-trifluorobenzyl (Z)-3-(2-cyanovinyl)-2,2-dimethylcyclopropanecarboxylate, represented by formula II:

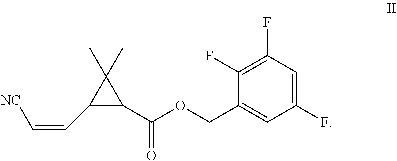 [SMILES: CC1(C)C(/C=C\C#N)C1C(=O)OCc1cc(F)cc(F)c1F]

The present invention further provides a method for synthesizing the pyrethroid compound containing a cyano group and a double bond, comprising the following steps:

### (1) Synthesizing a Z Configuration Product:

reacting t-butyl 3-formyl-2,2-dimethyl-cyclopropanoate with cyanomethyl phosphonate or cyanomethyl triphenylphosphine under the action of an alkali in solvent I to generate t-butyl 3-(2-cyanovinyl)-2,2-dimethyl-cyclopropanoate, followed by column chromatography to obtain t-butyl (Z)-3-(2-cyanovinyl)-2,2-dimethyl-cyclopropanoate;
   (2) Removing t-Butyl Group:
removing t-butyl group from t-butyl (Z)-3-(2-cyanovinyl)-2,2-dimethyl-cyclopropanoate obtained in step (1) under the action of an acid to obtain (Z)-3-(2-cyanovinyl)-2,2-dimethyl-cyclopropanoic acid;

### (3) Performing Acyl Chlorination Reaction:

subjecting (Z)-3-(2-cyanovinyl)-2,2-dimethyl-cyclopropanoic acid obtained in step (2) to an acyl chlorination reaction to obtain (Z)-3-(2-cyanovinyl)-2,2-dimethyl-cyclopropanoyl chloride;

### (4) Performing Esterification Reaction:

subjecting (Z)-3-(2-cyanovinyl)-2,2-dimethyl-cyclopropanoyl chloride obtained in step (3) and benzyl alcohol to an esterification reaction under the action of an alkali to obtain a pyrethroid compound containing a cyano group and a double bond.

The chemical reaction equation is as follows:

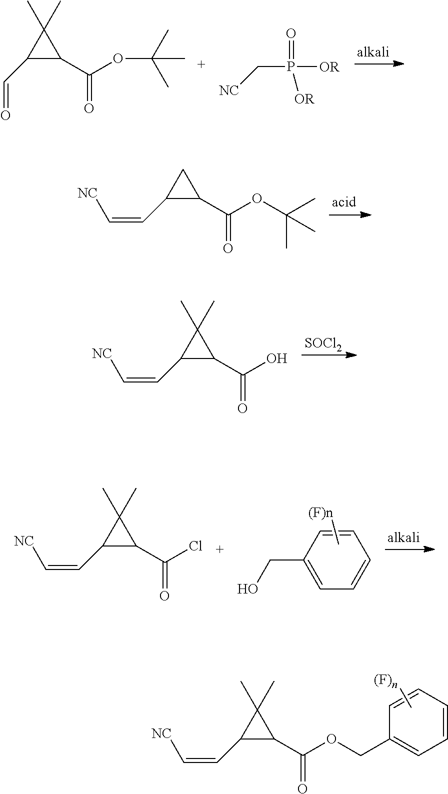

In the above technical solution, in step (1), the specific operation of synthesizing a Z configuration product is as follows: suspending an alkali in solvent I at −5 to −30° C., adding cyanomethyl phosphonate or cyanomethyl triphenylphosphine dropwise into the system at −5 to −30° C., then adding t-butyl 3-formyl-2,2-dimethyl-cyclopropanoate dropwise; maintaining the temperature at −5 to −30° C. for 1 h, and after gas chromatography (GC) detects complete conversion of the raw materials, using 10% hydrochloric acid to neutralize the system to a pH of 7 to 8, followed by extracting the aqueous layer with an extracting solvent to obtain organic phases, and the organic phases being subjected to desolvation and column chromatography to obtain t-butyl (Z)-3-(2-cyanovinyl)-2,2-dimethyl-cyclopropanoate.

In the above technical solution, in step (1), the cyanomethyl phosphonate is dimethyl cyanomethyl phosphonate, diethyl cyanomethyl phosphonate or isopropyl cyanomethyl phosphonate, preferably dimethyl cyanomethyl phosphonate.

In the above technical solution, in step (1), the molar ratio of the cyanomethyl phosphonate or cyanomethyl triphenylphosphine to t-butyl 3-formyl-2,2-dimethyl-cyclopropanoate is 0.5-1.5:1.

In the above technical solution, in step (1), the solvent I is any one of tetrahydrofuran, 2-methyltetrahydrofuran, acetonitrile, toluene, or a mixture of any two or more of the above mixed in any ratio, preferably tetrahydrofuran; the solvent I is used in an amount of 3 to 15 times the weight of t-butyl 3-formyl-2,2-dimethyl-cyclopropanoate.

In the above technical solution, in step (1), the alkali is any one of sodium methoxide, sodium ethoxide, sodium amide, sodium tert-butanol and potassium tert-butanol, or a mixture of any two or more of the above mixed in any ratio; the alkali is used in an amount of 1 to 5 molar equivalents of cyanomethyl phosphonate or cyanomethyl triphenylphosphine, preferably 1.2 molar equivalents.

In the above technical solution, in step (1), the extracting solvent is any one of ethyl acetate, methyl acetate, dichloromethane, dichloroethane, chloroform, toluene, xylene and benzene, or a mixture of any two or more of the above mixed in any ratio, preferably ethyl acetate; the extracting solvent is 1 to 5 times the weight of t-butyl 3-formyl-2,2-dimethyl-cyclopropanoate.

In the above technical solution, in step (2), the specific operation of removing t-butyl group is as follows: dissolving t-butyl (Z)-3-(2-cyanovinyl)-2,2-dimethyl-cyclopropanoate in solvent II, and removing t-butyl group by reaction under the catalytic action of an acid and at a normal pressure reflux temperature (61 to 140° C.) of the reactive solvent; after thin layer chromatography (TLC) detects complete conversion of the raw materials, cooling the system followed by washing with water to a pH of 6 to 7 and desolvation to obtain (Z)-3-(2-cyanovinyl)-2,2-dimethyl-cyclopropanoic acid.

In the above technical solution, in step (2), the solvent II is any one of toluene, xylene, benzene, dichloroethane, chloroform and tetrahydrofuran, or a mixture of any two or more of the above mixed in any ratio, preferably toluene; the solvent II is used in an amount of 3 to 10 times the weight of t-butyl (Z)-3-(2-cyanovinyl)-2,2-dimethyl-cyclopropanoate.

In the above technical solution, in step (2), the acid is any one of p-toluenesulfonic acid, methanesulfonic acid, hydrochloric acid and hydrobromic acid, or a mixture of any two or more of the above mixed in any ratio, preferably p-toluenesulfonic acid; the acid is used in an amount of 0.01 to 1 molar equivalents of t-butyl (Z)-3-(2-cyanovinyl)-2,2-dimethyl-cyclopropanoate, preferably 0.03 molar equivalents.

In the above technical solution, in step (3), the specific operation of the acyl chlorination reaction is as follows: dissolving (Z)-3-(2-cyanovinyl)-2,2-dimethyl-cyclopropanoic acid in solvent III, heating to a normal pressure reflux temperature (39 to 140° C.) of the reactive solvent, adding thionyl chloride dropwise under reflux conditions, and upon completion of the dropwise addition, maintaining the temperature at 10 to 60° C. for 0.5 to 2 h; following complete conversion of the raw materials under controlled conditions in the gas phase, performing desolvation to obtain (Z)-3-(2-cyanovinyl)-2,2-dimethyl-cyclopropanoyl chloride.

In the above technical solution, in step (3), the solvent III is any one of n-hexane, cyclohexane, methylcyclohexane, methylene chloride, chloroform, ethylene dichloride, benzene, toluene and xylene, or a mixture of any two or more of the above mixed in any ratio, preferably n-hexane; the solvent III is used in an amount of 3 to 10 times the weight of (Z)-3-(2-cyanovinyl)-2,2-dimethyl-cyclopropanoic acid.

In the above technical solution, in step (3), thionyl chloride is used in an amount of 1 to 3 molar equivalents of (Z)-3-(2-cyanovinyl)-2,2-dimethyl-cyclopropanoic acid, preferably 1.2 molar equivalents.

In the above technical solution, in step (4), the specific operation of the esterification reaction is as follows: dissolving benzyl alcohol in toluene and then adding an acid binding agent, adding (Z)-3-(2-cyanovinyl)-2,2-dimethyl-cyclopropanoyl chloride dropwise at 0 to 10° C., and upon completion of the dropwise addition, maintaining the temperature at 0 to 10° C. for 0.5 to 2 h; after complete conversion of the benzyl alcohol under controlled conditions in the gas phase, neutralizing the system with dilute hydrochloric acid to a pH of 6 to 7 followed by stratification, with organic phases being subjected to desolvation, column chromatographic separation and desolvation to obtain a pyrethroid compound containing a cyano group and a double bond.

In the above technical solution, in step (4), the benzyl alcohol is 2,3,6-trifluorobenzyl alcohol, 2,3,5-trifluorobenzyl alcohol or 2,3,5,6-tetrafluorobenzyl alcohol.

In the above technical solution, in step (4), the acid binding agent is triethylamine, pyridine or liquid alkali (aqueous solution of sodium hydroxide).

In the above technical solution, in step (4), the molar ratio of the benzyl alcohol, the acid binding agent and (Z)-3-(2-cyanovinyl)-2,2-dimethyl-cyclopropanoyl chloride is 0.9-1.2:1.0-1.3:1.

In the above technical solution, in step (4), the toluene is used in an amount of 3 to 10 times the weight of (Z)-3-(2-cyanovinyl)-2,2-dimethyl-cyclopropanoyl chloride.

The present invention further provides an application of the pyrethroid compound containing a cyano group and a double bond as an insecticide for prevention and control of sanitary pests.

In the above technical solution, when the pyrethroid compound containing a cyano group and a double bond is used as an insecticide for preventing and controlling sanitary pests, the pyrethroid compound containing a cyano group and a double bond represented by formula I is made into a mosquito repellent aerosol (the content is 0.3%) or an electric mosquito repellent incense (the content is 0.04%), for killing mosquitoes, flies and German cockroaches etc.

Compared with the prior art, the present invention provides a pyrethroid compound with a novel structure, which is slightly toxic to mammals. For example, the compound 2,3,5,6-tetrafluorobenzyl (Z)-3(2-cyanovinyl)-2,2-dimethylcyclopropanecarboxylate according to the present invention has a rat acute oral toxicity median lethal dose (LD50)>5000 mg/kg, classified as slightly toxic; and a rat acute dermal toxicity median lethal dose (LD50)>5000 mg/kg, also classified as slightly toxic. It exhibits significantly lower acute toxicity in rats than dimefluthrin and metofluthrin commonly available on the market, making it safer for non-target organisms, while demonstrating excellent effectiveness against some specific sanitary pests. In addition, it is characterized with high vapor pressure, and thus is suitable for use in various types of room-temperature volatile sanitary formulations.

### DETAILED DESCRIPTION OF THE INVENTION

The embodiments of the technical solution of the present invention are described in detail hereinafter, but the present invention is not limited to the following description: The raw materials in the Examples of the present invention, namely, t-butyl 3-formyl-2,2-dimethyl-cyclopropanoate, cyanomethyl phosphonate or cyanomethyl triphenylphosphine, 2,3,6-trifluorobenzyl alcohol, 2,3,5-trifluorobenzyl alcohol, 2,3,5,6-tetrafluorobenzyl alcohol and Momfluorothrin are commercially available.

The present invention is described hereinafter in conjunction with specific examples:

### Example 1

### (1) Synthesis of t-butyl (Z)-3-(2-cyanovinyl)-2,2-dimethyl-cyclopropanoate

In a 250 mL four-neck flask equipped with a stirrer, 3.51 g (90 mmol) sodium amide and 80 mL tetrahydrofuran were added, stirred and cooled to −20° C. Then, 6.7 g (45 mmol) dimethyl cyanomethyl phosphonate was added dropwise. Upon completion of the dropwise addition, the mixture was stirred for 15 min. At the above temperature, 8 g (40 mmol) t-butyl 3-formyl-2,2-dimethyl-cyclopropanoate was added dropwise. Upon completion of the dropwise addition, the mixture was maintained at the aforementioned temperature for 0.5 h. Following complete conversion of t-butyl 3-formyl-2,2-dimethyl-cyclopropanoate under controlled conditions in the gas phase, 5% hydrochloric acid was added dropwise until the system reached a pH of 7 to 8. Liquid separation was performed, with the aqueous layer extracted with 2*20 mL ethyl acetate, and the organic phases combined and desolvated, resulting in 8 g brown yellow liquid. Through column chromatography using ethyl acetate: n-hexane=1:10 (V/V), 4.32 g white solid t-butyl (Z)-3-(2-cyanovinyl)-2,2-dimethyl-cyclopropanoate was obtained, with a yield of 54%.

### (2) Synthesis of (Z)-3-(2-cyanovinyl)-2,2-dimethyl-cyclopropanoic acid

In a 250 mL four-neck flask equipped with a stirrer, 4.32 g (19.5 mmol) t-butyl (Z)-3-(2-cyanovinyl)-2,2-dimethyl-cyclopropanoate, 0.13 g p-toluenesulfonic acid and 25 mL toluene were added, stirred, heated to reflux at 110° C., and reacted under reflux for 1.5 h. TLC was employed to detect the reaction. Following complete conversion of the raw materials, the reaction mixture was cooled to room temperature, washed with water until the system reached a pH of 6 to 7, and desolvated to obtain 3 g light yellow solid (Z)-3-(2-cyanovinyl)-2,2-dimethyl-cyclopropanoic acid, with a yield of 93%.

### (3) Synthesis of (Z)-3-(2-cyanovinyl)-2,2-dimethyl-cyclopropanoyl chloride

In a 250 mL four-neck flask equipped with a stirrer, 3 g (18.2 mmol) (Z)-3-(2-cyanovinyl)-2,2-dimethyl-cyclopropanoic acid and 20 mL n-hexane were added, and 2 drops of DMF were added, stirred and heated to reflux. Then, 2.6 g (21.8 mmol) thionyl chloride was added dropwise. Upon completion of the dropwise addition, the mixture was maintained at the aforementioned temperature for 0.5 h. Following complete conversion of the raw materials under controlled conditions in the gas phase, desolvation was performed to obtain 3.2 g (17.5 mmol) dark green oily liquid (Z)-3-(2-cyanovinyl)-2,2-dimethyl-cyclopropanoyl chloride, with a yield of 96%.

### (4) Synthesis of 2,3,5-trifluorobenzyl (Z)-3-(2-cyanovinyl)-2,2-dimethylcyclopropanecarboxylate

In a 250 mL four-neck flask, 2.7 g (16.7 mmol) 2,3,5-trifluorobenzyl alcohol, 4.7 g (35 mmol) 30% aqueous solution of sodium hydroxide and 20 mL toluene were added, stirred and cooled to a temperature of 0 to 5° C. Then, 3.2 g (17.5 mmol) (Z)-3-(2-cyanovinyl)-2,2-dimethyl-cyclopropanoyl chloride was slowly added dropwise. Upon completion of the dropwise addition, the mixture was maintained at the aforementioned temperature. Following complete conversion of the benzyl alcohol under controlled conditions in the gas phase, the pH was adjusted to a range of 6 to 7 using hydrochloric acid, followed by stratification and desolvation of organic phases. Through column chromatographic separation using ethyl acetate: n-hexane=1:15 (V/V) and desolvation, 4.7 g colorless oily liquid 2,3,5-trifluorobenzyl (Z)-3-(2-cyanovinyl)-2,2-dimethylcyclopropanecarboxylate was obtained, with a yield of 92%.

### Example 2

### (1) Synthesis of t-butyl (Z)-3-(2-cyanovinyl)-2,2-dimethyl-cyclopropanoate

In a 250 mL four-neck flask equipped with a stirrer, 6.8 g (100 mmol) sodium ethoxide and 50 mL tetrahydrofuran were added, stirred and cooled to −20° C. Then, 8.95 g (50 mmol) diethyl cyanomethyl phosphonate was added dropwise. Upon completion of the dropwise addition, the mixture was stirred for 15 min. At the above temperature, 9 g (45 mmol) t-butyl 3-formyl-2,2-dimethyl-cyclopropanoate was added dropwise. Upon completion of the dropwise addition, the mixture was maintained at the aforementioned temperature for 0.5 h. Following complete conversion of the t-butyl 3-formyl-2,2-dimethyl-cyclopropanoate under controlled conditions in the gas phase, 5% hydrochloric acid was added dropwise until the system reached a pH of 7 to 8. Liquid separation was performed, with the aqueous layer extracted using 2*20 mL of ethyl acetate, and the organic phases combined and desolvated, resulting in 9.5 g brown yellow liquid. Through column chromatography using ethyl acetate: n-hexane=1:10 (V/V), 5.17 g white solid t-butyl (Z)-3-(2-cyanovinyl)-2,2-dimethyl-cyclopropanoate was obtained, with a yield of 52%.

### (2) Synthesis of (Z)-3-(2-cyanovinyl)-2,2-dimethyl-cyclopropanoic acid

In a 250 mL four-neck flask equipped with a stirrer, 5.17 g (23.4 mmol) t-butyl (Z)-3-(2-cyanovinyl)-2,2-dimethyl-cyclopropanoate, 0.16 g p-toluenesulfonic acid and 25 mL dichloroethane were added, stirred, heated to reflux at 84° C., and reacted under reflux for 6 h. TLC was employed to detect the reaction. Following complete conversion of the raw materials, the reaction mixture was cooled to room temperature, washed with water until the system reached a pH of 6 to 7, and desolvated to obtain 3.7 g light yellow solid (Z)-3-(2-cyanovinyl)-2,2-dimethyl-cyclopropanoic acid, with a yield of 96%.

### (3) Synthesis of (Z)-3-(2-cyanovinyl)-2,2-dimethyl-cyclopropanoyl chloride

In a 250 mL four-neck flask equipped with a stirrer, 3.7 g (22.4 mmol) (Z)-3-(2-cyanovinyl)-2,2-dimethyl-cyclopropanoic acid and 20 mL dichloromethane were added, and 2 drops of DMF were added, stirred and heated to reflux at 40° C. Then, 3.2 g (26.9 mmol) thionyl chloride was added. Upon completion of the dropwise addition, the mixture was maintained at the aforementioned temperature for 0.5 h. Following complete conversion of the raw materials under controlled conditions in the gas phase, desolvation was performed to obtain 4.16 g (20.8 mmol) dark green oily liquid (Z)-3-(2-cyanovinyl)-2,2-dimethyl-cyclopropanoyl chloride, with a yield of 93%.

### (4) Synthesis of 2,3,6-trifluorobenzyl (Z)-3-(2-cyanovinyl)-2,2-dimethylcyclopropanecarboxylate

In a 250 mL four-neck flask, 3.0 g (18.7 mmol) 2,3,6-trifluorobenzyl alcohol, 2.95 g (37.4 mmol) pyridine and 20 mL toluene were added, stirred and cooled to a temperature of 0 to 5° C. Then, 4.16 g (20.8 mmol) (Z)-3-(2-cyanovinyl)-2,2-dimethyl-cyclopropanoyl chloride was slowly added dropwise. Upon completion of the dropwise addition, the mixture was maintained at the aforementioned temperature. Following complete conversion of the benzyl alcohol under controlled conditions in the gas phase, the pH was adjusted to a range of 6 to 7 using hydrochloric acid, followed by stratification and desolvation of organic phases. Through column chromatographic separation using ethyl acetate: n-hexane=1:15 (V/V) and desolvation, 5.14 g colorless oily liquid 2,3,6-trifluorobenzyl (Z)-3-(2-cyanovinyl)-2,2-dimethylcyclopropanecarboxylate was obtained, with a yield of 89%.

### Example 3

### (1) Synthesis of t-butyl (Z)-3-(2-cyanovinyl)-2,2-dimethyl-cyclopropanoate

In a 250 mL four-neck flask equipped with a stirrer, 4.86 g (90 mmol) sodium methoxide and 60 mL acetonitrile were added, stirred and cooled to −20° C. Then, 6.7 g (45 mmol) dimethyl cyanomethyl phosphonate was added dropwise. Upon completion of the dropwise addition, the mixture was stirred for 15 min. At the above temperature, 8 g (40 mmol) t-butyl 3-formyl-2,2-dimethyl-cyclopropanoate was added dropwise. Upon completion of the dropwise addition, the mixture was maintained at the aforementioned temperature for 0.5 h. Following complete conversion of the t-butyl 3-formyl-2,2-dimethyl-cyclopropanoate under controlled conditions in the gas phase, 5% hydrochloric acid was added dropwise until the system reached a pH of 7 to 8. Liquid separation was performed, with the aqueous layer extracted using 2*20 mL ethyl acetate, and the organic phases combined and desolvated, resulting in 8.4 g brown yellow liquid. Through column chromatography using ethyl acetate: n-hexane=1:10 (V/V), 5.22 g white solid t-butyl (Z)-3-(2-cyanovinyl)-2,2-dimethyl-cyclopropanoate was obtained, with a yield of 59%.

### (2) Synthesis of (Z)-3-(2-cyanovinyl)-2,2-dimethyl-cyclopropanoic acid

In a 250 mL four-neck flask equipped with a stirrer, 5.22 g (23.6 mmol) t-butyl (Z)-3-(2-cyanovinyl)-2,2-dimethyl-cyclopropanoate, 0.16 g p-toluenesulfonic acid and 30 mL xylene were added, stirred, heated to reflux at 140° C., and reacted under reflux for 2 h. TLC was employed to detect the reaction. Following complete conversion of the raw materials, the reaction mixture was cooled to room temperature, washed with water until the system reached a pH of 6 to 7, and desolvated to obtain 3.66 g light yellow solid (Z)-3-(2-cyanovinyl)-2,2-dimethyl-cyclopropanoic acid, with a yield of 94%.

### (3) Synthesis of (Z)-3-(2-cyanovinyl)-2,2-dimethyl-cyclopropanoyl chloride

In a 250 mL four-neck flask equipped with a stirrer, 3.66 g (22.2 mmol) (Z)-3-(2-cyanovinyl)-2,2-dimethyl-cyclopropanoic acid and 20 mL cyclohexane were added, and 2 drops of DMF were added, stirred and heated to reflux at 81° C. Then, 3.96 g (33.3 mmol) thionyl chloride was added. Upon completion of the dropwise addition, the mixture was maintained at the aforementioned temperature for 0.5 h. Following complete conversion of the raw materials under controlled conditions in the gas phase, desolvation was performed to obtain 4.34 g (21.76 mmol) dark green oily liquid (Z)-3-(2-cyanovinyl)-2,2-dimethyl-cyclopropanoyl chloride, with a yield of 98%.

### (4) Synthesis of 2,3,5,6-tetrafluorobenzyl (Z)-3(2-cyanovinyl)-2,2-dimethylcyclopropanecarboxylate

In a 250 mL four-neck flask, 3.33 g (18.5 mmol) 2,3,5,6-tetrafluorobenzyl alcohol, 4.7 g (35 mmol) aqueous solution of sodium hydroxide and 20 mL toluene were added, stirred and cooled to a temperature of 0 to 5° C. Then, 4.34 g (21.76 mmol) (Z)-3-(2-cyanovinyl)-2,2-dimethyl-cyclopropanoyl chloride was slowly added dropwise. Upon completion of the dropwise addition, the mixture was maintained at the aforementioned temperature. Following complete conversion of the benzyl alcohol under controlled conditions in the gas phase, the pH was adjusted to a range of 6 to 7 using hydrochloric acid, followed by stratification and desolvation of organic phases. Through column chromatographic separation using ethyl acetate: n-hexane=1:20 (V/V) and desolvation, 5.32 g colorless oily liquid 2,3,5,6-tetrafluorobenzyl (Z)-3(2-cyanovinyl)-2,2-dimethylcyclopropanecarboxylate was obtained, with a yield of 88%.

The pyrethroid compounds 1-3 containing a cyano group and a double bond of the present invention prepared according to the synthesis methods of Examples 1-3 are represented by formula I:

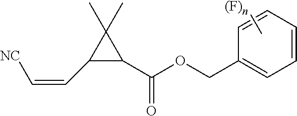

### Formula I

wherein n is 3 or 4.

TABLE 1
List of compounds 1-3 of the Present invention and Control compound 4
Momfluorothrin

| Com- | |
|---|---|
| pound | |
| No. | Formula |
| 1 | 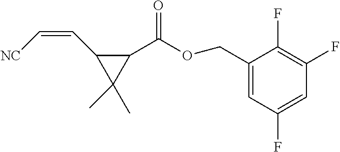 [SMILES: CC1(C)C(/C=C\C#N)C1C(=O)OCc1cc(F)cc(F)c1F] |
| 2 | 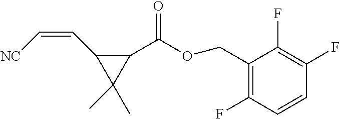 [SMILES: CC1(C)C(/C=C\C#N)C1C(=O)OCc1c(F)ccc(F)c1F] |
| 3 | 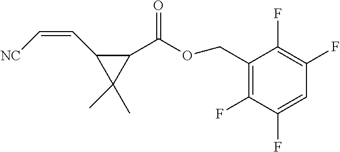 [SMILES: CC1(C)C(/C=C\C#N)C1C(=O)OCc1c(F)c(F)cc(F)c1F] |
| 4 | 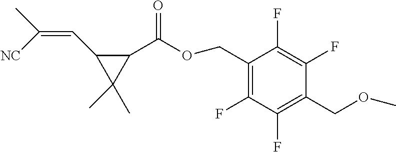 [SMILES: COCc1c(F)c(F)c(COC(=O)C2C(/C=C(/C)C#N)C2(C)C)c(F)c1F] |

The following test examples demonstrate that the pyrethroid compound of the present invention is effective as a sanitary pest control agent.

### Test Example 1

99.96 parts by weight of a mixture consisting of corn starch, carbon powder and wood powder (1:5:4) were added with 120 parts by mass of water, kneaded into shape, and then dried to form a mosquito-repellent incense substrate (with a diameter of 12.0 cm, a thickness of 4 mm, and a pair weighing 40 g).

On the other hand, a 0.4 w/v % solution of compound 1 in kerosene was prepared. The set of incense substrate was uniformly spray-coated with 4 ml of the above solution using a microsyringe, and then left to dry at room temperature for 3 h to obtain a set of mosquito-repellent incense W1 containing 0.04 w/w % compound 1.

Similarly, compounds 2-3 of the present invention shown in Table 1, as well as Momfluorothrin and tetramethylfluthrin were prepared respectively to obtain corresponding mosquito-repellent incenses W2 to W5.

The mosquito-repellent incenses W2 to W5 were measured and compared in terms of mosquito-killing efficacy in accordance with GB/T13917.4-2009. The test insects were female mosquitoes of Culex pipiens pallens, which did not suck blood for 2 to 3 days after eclosion. The specific process was as follows: sucking 20 test mosquitoes with a mosquito suction tube, placing them into a sealed drum apparatus, taking a section of mosquito-repellent incense to be tested and placing it on an incense holder, lighting the incense and starting timing, removing the mosquito-repellent incense after 1 min, and recording the number of test mosquitos knocked down at regular intervals. The experimental results are shown in Table 2, demonstrating that compounds 1-3 of the present invention synthesized by simplifying the structure of Momfluorothrin exhibited a significantly better mosquito-repellent efficacy than Momfluorothrin, and was remarkably superior to control compound, commercialized pyrethrin-tetrafluoromethyl ether.

TABLE 2
Comparison of Some Compounds of the Present Invention and
the Control Compound in terms of Mosquito-Killing Effect

| | | Concen- | |
|---|---|---|---|
| | Active | tration | KT50 |
| Mosquito-repellent incense | ingredients | w/w % | (min) |
| Mosquito-repellent incense W1 | Compound 1 | 0.04 | 2.45 |
| Mosquito-repellent incense W2 | Compound 2 | 0.04 | 5.42 |
| Mosquito-repellent incense W3 | Compound 3 | 0.04 | 4.65 |
| Mosquito-repellent incense W4 | Momfluorothrin | 0.04 | 27.87 |
| Mosquito-repellent incense W5 | Control compound | 0.04 | 9.23 |
| | tetramethylfluthrin | | |

### Test Example 2

0.3 parts by weight of compound 1 with 59.7 parts by weight of kerosene were heated and evenly mixed to prepare an insecticide. The resulting preparation was placed in an aerosol can equipped with a valve, through which 40.0 parts by weight of propane and butane were injected underpressure, to obtain an insecticidal aerosol containing 0.3% compound 1.

The insecticidal aerosol was tested for efficacy on mosquitoes, flies, and German cockroaches in accordance with GB/T13917.2-2009, using a sealed drum apparatus. The specific process was as follows: placing the test insects into a tank, and after the test insects resumed normal activities, quantitatively spraying 1 g of the agent from the insecticidal aerosol can, pulling out the baffle after 1 min to make the test insects in contact with the agent, immediately starting timing and recording the number of the test insects knocked down at regular intervals, transferring all the test insects to a clean insect rearing cage after 20 min, and checking the number of dead insects after 24 h, wherein the German cockroaches were tested for 72-h mortality rate.

The aerosols of compounds 1-3, Momflurothrin and tetramethylfluthrin were prepared according to Test Example 2, and were tested for efficacy. The comparison results are shown in Table 3.

TABLE 3
Killing Effects of Insecticidal Aerosols Prepared from Various
Compounds on Mosquitoes, Flies and German Cockroaches

| | Active ingredient of | | Mortality | |
|---|---|---|---|---|
| Target | aerosol | KT50(min) | rate | |
| Mosquitoes | Compound 1 | 1.33 | 100% | (24 h) |
| | Compound 2 | 3.58 | 100% | (24 h) |
| | Compound 3 | 2.45 | 100% | (24 h) |
| | Momfluorothrin | 12.35 | 100% | (24 h) |
| | tetramethylfluthrin | 6.46 | 100% | (24 h) |
| Flies | Compound 1 | 2.32 | 100% | (24 h) |
| | Compound 2 | 4.65 | 100% | (24 h) |
| | Compound 3 | 3.45 | 100% | (24 h) |
| | Momfluorothrin | 15.28 | 70% | (24 h) |
| | tetramethylfluthrin | 8.56 | 80% | (24 h) |
| German | Compound 1 | 4.05 | 95% | (72 h) |
| cockroaches | Compound 2 | 6.26 | 90% | (72 h) |
| | Compound 3 | 5.52 | 95% | (72 h) |
| | Momfluorothrin | 17.56 | 60% | (72 h) |
| | tetramethylfluthrin | 9.12 | 65% | (72 h) |

The results showed that the aerosol prepared from compound 1 of the present invention had relatively good prevention and control effect on mosquitoes, flies and German cockroaches, and was better than other compounds in terms of effect.

The above examples are only for illustrating the technical concept and technical features of the present invention, and are not intended to limit the protection scope of the present invention. All equivalent transformations or modifications made based on the substance of the present invention should fall within the protection scope of the present invention.

1. A pyrethroid compound containing a cyano group and a double bond, represented by formula I: 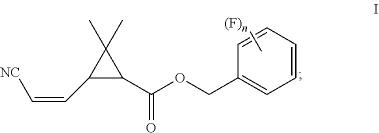
wherein n is 3 or 4.

2. The pyrethroid compound according to claim 1, wherein when n is 3, the compound is 2,3,5-trifluorobenzyl (Z)-3-(2-cyanovinyl)-2,2-dimethylcyclopropanecarboxylate or 2,3,6-trifluorobenzyl (Z)-3-(2-cyanovinyl)-2,2-dimethylcyclopropanecarboxylate.

3. The pyrethroid compound according to claim 1, wherein when n is 4, the compound is 2,3,5,6-tetrafluorobenzyl (Z)-3(2-cyanovinyl)-2,2-dimethylcyclopropanecarboxylate.

4. The pyrethroid compound according to claim 1, wherein the compound is 2,3,5-trifluorobenzyl (Z)-3-(2-cyanovinyl)-2,2-dimethylcyclopropanecarboxylate, represented by formula II:

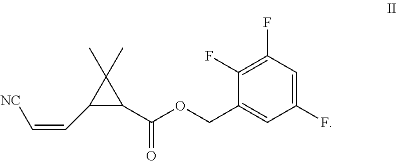 [SMILES: CC1(C)C(/C=C\C#N)C1C(=O)OCc1cc(F)cc(F)c1F]

5. A method for synthesizing the pyrethroid compound containing a cyano group and a double bond according to claim 1, comprising the following steps:
(1) synthesizing a Z configuration product:
reacting t-butyl 3-formyl-2,2-dimethyl-cyclopropanoate with cyanomethyl phosphonate or cyanomethyl triphenylphosphine under the action of an alkali in solvent I to generate t-butyl 3-(2-cyanovinyl)-2,2-dimethyl-cyclopropanoate, followed by column chromatography to obtain t-butyl (Z)-3-(2-cyanovinyl)-2,2-dimethyl-cyclopropanoate;
(2) removing t-butyl group:
removing t-butyl group from t-butyl (Z)-3-(2-cyanovinyl)-2,2-dimethyl-cyclopropanoate obtained in step (1) under the action of an acid to obtain (Z)-3-(2-cyanovinyl)-2,2-dimethyl-cyclopropanoic acid;
(3) performing acyl chlorination reaction:
subjecting (Z)-3-(2-cyanovinyl)-2,2-dimethyl-cyclopropanoic acid obtained in step (2) to an acyl chlorination reaction to obtain (Z)-3-(2-cyanovinyl)-2,2-dimethyl-cyclopropanoyl chloride;
(4) performing esterification reaction:
subjecting (Z)-3-(2-cyanovinyl)-2,2-dimethyl-cyclopropanoyl chloride obtained in step (3) and benzyl alcohol to an esterification reaction under the action of an alkali to obtain a pyrethroid compound containing a cyano group and a double bond.

6. The synthesis method according to claim 5, wherein in step (1), an alkali is suspended in solvent I at −5 to −30° C., cyanomethyl phosphonate or cyanomethyl triphenylphosphine are first added dropwise at −5 to −30° C., and t-butyl 3-formyl-2,2-dimethyl-cyclopropanoate is then added dropwise, for reaction to obtain t-butyl (Z)-3-(2-cyanovinyl)-2,2-dimethyl-cyclopropanoate; the solvent I is any one of tetrahydrofuran, 2-methyltetrahydrofuran, acetonitrile and toluene, or a mixture of any two or more of the above mixed in any ratio.

7. The synthesis method according to claim 5, wherein in step (2), t-butyl (2)-3-(2-cyanovinyl)-2,2-dimethyl-cyclopropanoate is dissolved in solvent II, and under the catalytic action of an acid, (Z)-3-(2-cyanovinyl)-2,2-dimethyl-cyclopropanoic acid is obtained; the solvent II is any one of toluene, xylene, benzene, dichloroethane, chloroform and tetrahydrofuran, or a mixture of any two or more of the above mixed in any ratio.

8. The synthesis method according to claim 5, wherein in step (3), (Z)-3-(2-cyanovinyl)-2,2-dimethyl-cyclopropanoic acid is dissolved in solvent Ill, and thionyl chloride is added dropwise under normal pressure reflux conditions, for reaction to obtain (Z)-3-(2-cyanovinyl)-2,2-dimethyl-cyclopropanoyl chloride; the solvent Ill is any one of n-hexane, cyclohexane, methylcyclohexane, methylene chloride, chloroform, ethylene dichloride, benzene, toluene and xylene, or a mixture of any two or more of the above mixed in any ratio.

9. The synthesis method according to claim 5, wherein in step (4), benzyl alcohol is dissolved in toluene, then an acid binding agent is added, and (Z)-3-(2-cyanovinyl)-2,2-dimethyl-cyclopropanoyl chloride is added dropwise under the conditions of 0 to 30° C., for reaction to obtain the pyrethroid compound containing a cyano group and a double bond of formula I; the acid binding agent is triethylamine, pyridine or liquid alkali.

10. An application of the pyrethroid compound containing a cyano group and a double bond according to claim 1 as an insecticide for prevention and control of sanitary pests, wherein the pyrethroid compound containing a cyano group and a double bond represented by formula I is made into a mosquito repellent aerosol or an electric mosquito repellent, for killing mosquitoes, flies and German cockroaches.
